(19) United States
(12) Patent Application Publication
(10) Pub. No.: US 2004/0186426 A1
(43) Pub. Date: Sep. 23, 2004

(54) Blood collecting syringe with retractable needle

(76) Inventor: Edward F. Allard, Springfield, VA (US)

Correspondence Address: EDWARD F. ALLARD, 7830 GREELEY BLVD, SPRINGFIELD, VA, 22152, US

(21) Appl. No.: 10/392,181

(22) Filed: Mar. 20, 2003

(51) Int. Cl. A61M 5/00

(52) U.S. Cl. 604/110; 600/577

(57) ABSTRACT

A blood-taking hypodermic syringe having a needle assembly which, after removing a vial of drawn blood, may be retracted into the housing of the syringe, in such a way that the needle becomes inaccessible, is rendered non-operational and the housing becomes a safe storage container for the spent needle and for any residual fluids.

### BACKGROUND OF THE INVENTION

1. Field of the Invention

This invention relates to blood-taking syringes, and more particularly to a syringe having a needle assembly which, after removing a vial of drawn blood, may be retracted into the housing of the syringe, in such a way that the needle becomes inaccessible, is rendered non-operational and the housing becomes a safe storage container for the spent needle and for any residual fluids.

2. Description of the Prior Art

The spread of diseases to a caregiver, from the inadvertent exposure to contaminated needles or body fluids, resulting from the use of syringes, has become a major concern throughout the health industry. Many attempts have been made to reduce the likelihood of the user being exposed to needle pricks and residual body fluids, by designing various schemes to cap the spent needle or to retract the needle into the cavity of the syringe, but in essentially all instances, the syringe has failed to provide an easily actuated mechanism for retracting and safely storing the needle in a fail-safe container that can then be safely handled without fear of being exposed to the spent needle and collected fluids. The instant technique and apparatus for collecting blood and safely disposing of the spent needle fully meets the existent needs of the health care worker and effectively overcomes all the safety concerns of the prior art devices.

### SUMMARY OF THE INVENTION

The general purpose of this invention is to provide an operationally easy and safe way to retract the needle assembly of a blood taking syringe into the syringe housing to prevent the device from being reused, and then to seal the housing, rendering it a safe storage container for the needle assembly.

### BRIEF DESCRIPTION OF DRAWINGS

FIG. 1 shows a cut-away view of a blood-taking syringe as envisioned by this invention.

FIG. 2 shows the needle assembly of the blood-taking syringe in a retracted state.

FIG. 3a depicts a blood-taking syringe wherein the needle assembly restraining means is a disc which restrains the needle assembly by exerting a restraining force on the top surface of the needle assembly;

FIG. 3b and FIG. 3c depict typical restraining discs as envisioned by this invention.

### DESCRIPTION OF THE PREFERRED EMBODIMENTS

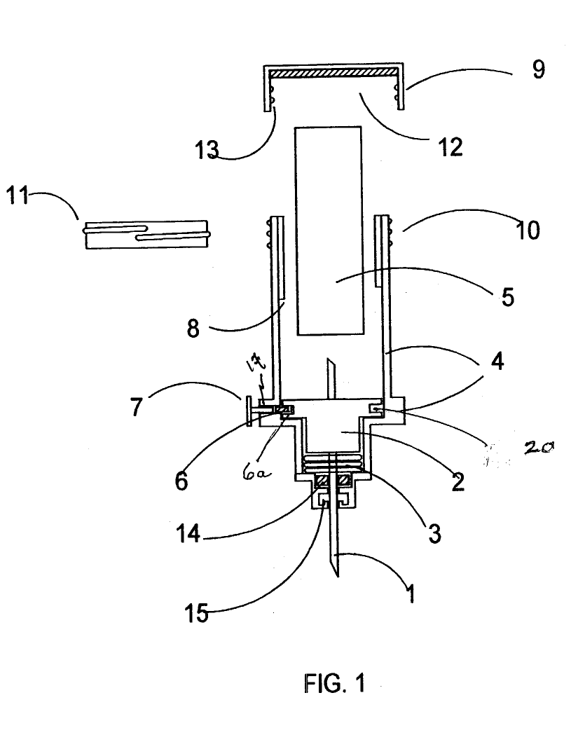

This invention will be best understood by referring to the drawings. Looking first at the blood taking syringe of FIG. 1, note the syringe housing (4), having first and second chambers, (4a) and (4b) respectively, with the first chamber (4a) adapted to receive a blood taking vacuum vial (5) and the second chamber (4b) designed to hold a spring loaded, double ended needle (1a) within the needle retainer (1b) of a hypodermic needle assembly (2). The double ended needle (1a) extends through the needle retainer (1b) and firmly holds needle (1a) in axial alignment with the central axis of the syringe housing (4), such that the distal end of the needle may be injected into a vein of a blood giver while the other end is axially aligned with the central axis of chamber (4a), and is so positioned that one end of the vacuum vial (5) will be punctured as an operator inserts the vacuum vial (5) into chamber (4a) of the syringe housing (4). As is well known in the art, the vacuum within the vacuum vial (5) then draws blood from the vein of the patient and fills the vial accordingly. Any desired number of vials of blood may be drawn from the patient before initiating the withdrawal of the needle from the patient and releasing the needle assembly (2) for storage within chamber (4a) of the syringe housing (4). A compressed spring (3) applies force to the needle assembly (2), attempting to force the needle assembly (2) into the inside of chamber (4a) of the syringe housing (4), however, spring (3) is prevented from driving the needle assembly into chamber (4a) by a needle assembly restraint means, here shown as a holding wedge (6), and accordingly holds the needle assembly (2) in a state of equilibrium. The holding wedge (6) can be fabricated of any of various materials, such as hard plastic or vinyl or, in fact, any material strong enough to withstand the force applied to the needle head assembly (2) by the compressed spring (3). The holding wedge may, optionally, be spring loaded, to assure the locking of the needle assembly (2) in a state of equilibrium, until the expressed depression of the release pin (7). A spring (6a), such as a leaf spring, may be either inserted into the groove (20) of the needle assembly (2) or actually attached to the spring during the manufacture of the syringe. After the desired number of vials of blood are drawn, the last vial may be removed from the container, and retraction of the needle assembly (2) may then be effected by depressing the needle assembly restraint release pin (7), which forces the end of pin (7) into the gap between the container holder (chamber 4a) and the needle head assembly (2), making contact with the edge of the holding wedge (6), forcing wedge (6) into groove (20) of the needle assembly (2) and releasing the force of the compressed spring (3) holding down the needle assembly (2). Depressing the release pin (7), by the thumb or forefinger, can be performed easily with the action of one hand. Even though not shown, another release pin and holding wedge may be used on the opposite side of the syringe housing from wedge (6), allowing the two release pins to be squeezed simultaneously between the thumb and forefinger to dislodge the holding wedges and release the needle assembly (2). The spring then would be free to expand and force the needle assembly (2) into chamber (4a) of syringe housing (4) until it is stopped by a protrusion on the inside of the housing (4), shown as a stop (8). This stop may be molded into the syringe housing (4) at manufacture or may be a simple sleeve inserted within the housing. Stop (8) may have a beveled edge at its most inwardly extending end, which functions as a stop for halting the movement of the needle assembly (2), during the retraction of the needle assembly into chamber (4a), while the sleeve further functions to provide a guide for vacuum vial (5) during the insertion of the vial into the first chamber (4a) of the syringe housing (4). Once the needle assembly (2) is retracted into the syringe housing (4), it cannot be reused. A safety cap (9) may be attached to the syringe housing, which is here shown as a screw-on type, but may be of any design that would securely seal the cap when applied to the container. In the particular case at hand, cap (9) is twisted onto guide (10) and may be similar to a push down, commercial safety cap used on safe medication bottles for protecting children. Removal of the cap is not intended once the needle assembly is retracted into the body of the syringe and cap (9) is attached. The safety cap has a seal (12), within the inner surface of the cap, which, upon attaching the cap, seals in any residual fluids remaining within chamber (4a) of housing (4). Rings (13) on cap (9) mate with guide (10) on the syringe housing (4), upon twisting cap (9) onto guide (10). Upon attaching the cap (9) to the outermost end of chamber (4a), the syringe housing (4) Meals the end of chamber (4a) of housing (4), thus eliminating the possibility of spilling contaminated fluid. Seal (14) is self-sealing and immediately closes the hole left by the needle (1a) upon retraction. Seal (14) then seals the outermost end of the second chamber (4b) of the distal end of the syringe housing (4), which prevents any inside contaminated fluid, that may be in chamber (4b), from leaking to the outside of the syringe and also prevents the retracted needle (1a) from protruding back through seal (12), through which the needle (1a) has been retracted. Cavity (15) captures any blood that might be wiped off needle (1a) by seal (14) during retraction of the needle (1a), however, an additional cap (not shown), similar to that of cap (9), may be used on the outermost end of the second chamber (4b) of the syringe housing (4), in the event that one desires to be assured that the spent needle and other body fluids are absolutely encapsulated within the housing upon retraction of the needle assembly.

Upon retraction of the needle assembly (2) into chamber (4a) of the syringe housing (4) and upon attaching safety caps to the outermost ends of chambers (4a) and (4b) of the syringe housing (4), the needle assembly (2) and any contaminated fluids within the housing (4) of the syringe are encapsulated within the syringe housing (4), thereby rendering the syringe housing (4) a safe container for users and handlers who may come in contact with the used syringe.

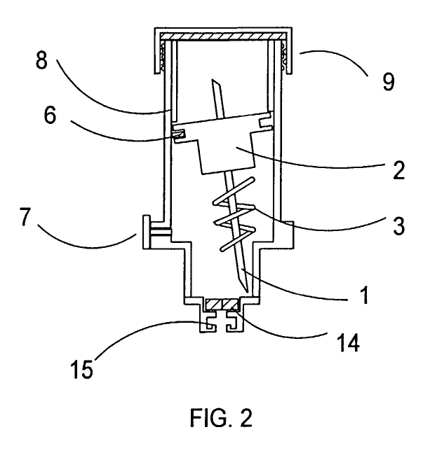

FIG. 2 shows the blood-taking device of FIG. 1 in its retracted state. After depressing the release pin(s) (7) and making contact with wedge (6), the wedge (6) is dislodged from the slot in the protrusion on the syringe housing to allow the needle assembly (2) to be retracted into the first chamber of the syringe housing and to be forced into groove (20) in the needle assembly (2). The double ended hypodermic needle (1a) has been retracted into chamber (4a), halted by stop (8) and twisted off the center axis of the syringe housing (4). Release pin (7) has been pushed in, forcing wedge (6) into a groove (20) in the needle assembly (2), which has released spring (3). Seal (14), being self-sealing, automatically closes upon retraction of the needle (1a), thereby sealing the second end portion (4b) of the syringe housing (4) from expelling any body fluids from the first portion (4a) of the housing and preventing the needle (1a) from inadvertently reentering the sealed hole in seal (14) Safety cap (9) is shown twisted onto the top of the syringe housing (4), which now completely seals the top of the first portion (4a) of the housing The needle and any residual body fluids are now inaccessibly stored in the housing of the syringe in such a manner that the syringe cannot be reused and the syringe becomes a safe storage container for the spent needle and fluids.

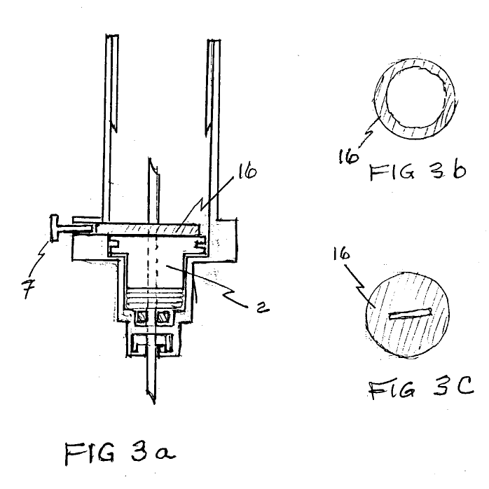

Looking now at FIG. 3a, one sees that the needle assembly restraining means (6) in FIG. 2, sitting within groove (20) of the needle assembly (2), now sits atop the needle assembly (2) instead of in the groove (20) and needle assembly (2) takes the form of a circular disc, such as an actual disc as shown in FIG. 3c having a slit cut therein to provide for passage of the needle (1a) therethrough, or alternatively, an annular ring as shown in FIG. 3c. In either case upon deactivating the needle assembly restraining means (16) by depressing the release pin (7), the needle assembly (2), as well as the needle assembly restraining means (16) is retracted into the upper chamber of the syringe housing, as described relative to the syringe of FIG. 1.

While the invention has been described in terms of two preferred embodiments, those skilled in the art will recognize that the invention can be readily practiced with modification within the spirit and scope of the appended claims.

Having thus described my invention, what I claim as new and desire to secure by Letters Patent is as follows:

1. A blood taking syringe comprising:
A syringe housing having a first chamber adapted to accept a removable blood-collecting vacuum vial and a second chamber adapted to hold a releasably mounted needle assembly under tension;
A needle assembly, including a cylindrically shaped needle retainer, having first and second ends with a double-ended hypodermic needle extending through the center thereof, with said needle and needle retainer axially aligned with the central axis of the syringe housing;
A compressable spring, normally held in a compressed mode, seated in the second chamber of the syringe housing, for applying pressure along the central axis of the syringe housing to the first end of the needle retainer;
A needle assembly restraining means for applying a restraining force to the second end of said needle retainer for maintaining said needle assembly in a state of equilibrium;
Needle assembly restraint release means for disengaging the needle assembly restraining means to allow said needle assembly to be retracted into the first chamber of the syringe housing.

2. The blood taking syringe of claim 1, wherein the needle retainer has a circumferential groove extending around the outer periphery of the second end thereof for accepting the needle assembly restraining means and restraining the needle assembly to maintain same in a state of equilibrium.

3. The blood taking syringe of claim 2, further including an outwardly extending protrusion on the syringe housing at the intersection of the first and second chambers of said housing, with said protrusion having an inwardly open slot for accepting a portion of the needle assembly restraining means, to allow the needle assembly restraining means to be interposed between the groove in the second end of the needle retainer and the slot in the syringe protrusion to hold the needle assembly in a state of equilibrium until the needle assembly restraint release means is engaged to release the needle assembly for retraction into the first chamber of the syringe.

4. The blood taking syringe of claim 3, wherein the needle assembly restraining means is a dislodgable wedge interposed within the groove of the needle retainer and the slot in the syringe protrusion and aligned with the restraint release means to allow the restraint release means to deactivate the restraint means upon demand.

5. The blood taking syringe of claim 4, wherein the groove around the periphery of the second end of the needle retainer is of sufficient depth to accept the entire wedge upon disengagement from the slit in the syringe protrusion and whereby the slot in the protrusion of the syringe housing is of sufficient depth to accept at least one half the width of the wedge; whereby the wedge may be fully seated within the slot of the protrusion of the syringe housing while simultaneously extending into the groove of the needle retainer to hold the needle assembly in a state of equilibrium until disengaged.

6. The blood taking syringe of claim 5, wherein at least one hole extends through the wall of the housing, at the site of the slot of the syringe protrusion, for receiving the restraint release means, which upon activation, forces the portion of the dislodgable wedge, lying within the slot in the protrusion of the syringe housing, fully into the groove of the needle retainer, to release the needle assembly for retraction into the first chamber of the syringe housing.

7. The blood taking syringe of claim 6, wherein the restraint release means is a manually activated push pin.

8. The blood taking syringe of claim 1, wherein the second chamber of the syringe housing is further adapted to seat a seal into which a self-sealing seal is inserted, through which the distal end of the double ended needle extends, such that upon drawing blood, removing the vacuum vial and retracting the needle and needle assembly into the first chamber of the syringe housing: the needle is retracted from the seal, allowing the resulting hole within the seal to close, whereby the distal end of the syringe is sealed, thereby preventing the leakage of any residual fluids within the body of the syringe to the outside of the syringe and effectively locking the needle assembly within the first chamber of the syringe.

9. The blood taking syringe of claim 3, wherein the second chamber of the syringe housing is further adapted to seat a seal into which a self-sealing seal is inserted, through which the distal end of the double ended needle extends, such that upon drawing blood, removing the vacuum vial and retracting the needle and needle assembly into the first chamber of the syringe housing the needle is retracted from the seal, allowing the resulting hole within the seal to close, whereby the distal end of the syringe is sealed, thereby preventing the leakage of any residual fluids within the body of the syringe to the outside of the syringe and effectively locking the needle assembly within the first chamber of the syringe.

10. The blood taking syringe of claim 6, wherein the second chamber of the syringe housing is further adapted to seat a seal into which a self-scaling scal is inserted, through which the distal end of the double ended needle extends, such that upon drawing blood, removing the vacuum vial and retracting the needle and needle assembly into the first chamber of the syringe housing, the needle is retracted from the seal, allowing the resulting hole within the seal to close, whereby the distal end of the syringe is scaled, thereby preventing the leakage of any residual fluids within the body of the syringe of the outside of the syringe and effectively locking the needle assembly within the first chamber of the syringe.

11. The blood taking syringe of claim 6, wherein the second chamber of the syringe housing is further adapted to seat a seal, into which a self-sealing seal is inserted, through which the distal end of the double ended needle extends, such that upon drawing blood, removing the vacuum vial and retracting the needle and needle assembly into the first chamber of the syringe housing, the distal end of the syringe is scaled against the leakage of any fluids remaining within the body of the syringe.

12. The blood taking syringe of claim 1, filter having a fluid collecting cavity located below the self-sealing seal, on the distal end of the syringe, which functions to collect any residual fluid wiped from the surface of the needle as the needle is retracted through the self-sealing seal into the first chamber of the syringe housing, thereby preventing any residual fluid from leaking to the outside of the housing.

13. The blood taking syringe of claim 3, further having a fluid collecting cavity located below the self-sealing seal, on the distal end of the syringe, which functions to collect any residual fluid wiped from the needle as the needle is retracted through the self-sealing seal into the first chamber of the syringe housing, thereby preventing any residual fluid from leaking to the outside of the housing.

14. The blood taking syringe of claim 6, further having a fluid collecting cavity located below the self-sealing seal, on the distal end of the syringe, which functions to collects any residual fluid wiped from the needle as the needle is retracted through the self-sealing seal into the first chamber of the syringe housing, thereby preventing any residual fluid from leaking to the outside of the housing.

15. The blood taking syringe of claim 11, further having a fluid collecting cavity located below the self-sealing seal, on the distal end of the syringe, which functions to collect any residual fluid wiped from the needle as the needle is retracted through the self-sealing seal into the first chamber of the syringe housing, thereby preventing any residual fluid from leaking to the outside of the housing.

16. The blood taking syringe of claim 1, further having safety cap means for sealing both chambers of the syringe housing from the outside environment prior to use of the syringe and for resealing same after use and retraction of the needle assembly into the first chamber of the syringe housing.

17. The blood taking syringe of claim 11, further having safety cap means for sealing both chambers of the syringe housing from the outside environment prior to use of the syringe and for resealing same after use and retraction of the needle assembly into the first chamber of the syringe housing.

18. The blood taking syringe of claim 15, further having safety cap means for sealing both chambers of the syringe housing from the outside environment prior to use of the syringe and for resealing same after use and retraction of the needle assembly into the first chamber of the syringe housing.

19. The blood taking syringe of claim 1, wherein the needle assembly restraining means is disc shaped with a center hole sufficiently large to allow one end of said needle to unobstructively pass therethrough, and whereupon the disc shaped needle assemble restraining means sits atop the needle assembly, applies a restraining force to the second end of the needle retainer and coorporates with the inner wall of the syringe housing to effectively lock the needle assembly into a state of equilibrium, until such time as is deemed appropriate to deactivate the needle restraining means to allow the needle assembly to be retracted into the housing of the syringe.

20. The blood taking syringe of claim 3, further containing a needle assembly stop means, which is a cylindrically shaped protrusion extending inwardly from the inside wall of the first chamber of the syringe housing while extending from the outermost end of the first chamber of the syringe, along the inside of the first chamber wall, for a distance that allows the end of the protrusion extending into the chamber to make contact with at least one edge of the needle retainer, as the needle retainer is being retracted into the first chamber, to arrest the retraction of the needle assembly at a point where the end of the double needle, used to puncture the vacuum vial, does not extend past the outermost end of the chamber, and whereby the resulting inside diameter of the chamber, in the portion having the protrusion therein, is less than the diameter of the cylindrical needle retainer, thereby preventing the needle retainer and thus the end of the double needle, used to puncture the vacuum vial, from extending past the outermost end of the first chamber of the syringe housing and whereby the inside protrusion in the first chamber of the syringe further provides a guide for the blood taking vacuum vial during the insertion of the vial into the syringe.
